(19) United States
(12) Patent Application Publication
(10) Pub. No.: US 2018/0153387 A1
(43) Pub. Date: Jun. 7, 2018

(54) LUBRICANT APPLICATOR DEVICE AND METHOD OF AN INSERTION TUBE OF AN ENDOSCOPE

(71) Applicant: Dr. Prosper Abitbol, Boca Raton, FL (US)

(72) Inventor: Dr. Prosper Abitbol, Boca Raton, FL (US)

(21) Appl. No.: 15/835,404

(22) Filed: Dec. 7, 2017

Related U.S. Application Data
(60) Provisional application No. 62/430,932, filed on Dec. 7, 2016.
(60) Provisional application No. 62/433,321, filed on Dec. 13, 2016.
(60) Provisional application No. 62/434,397, filed on Dec. 14, 2016.

(51) Int. Cl. A61B 1/12; A61B 1/04; A61B 1/06; A61B 1/005; A61B 1/00

(52) U.S. Cl. CPC A61B 1/12; A61B 1/04; A61B 1/00045; A61B 1/005; A61B 1/06

(57) ABSTRACT

Disclosed are a system, a device, and a method of a lubricant applicator device to be used with an insertion tube of an endoscope. The lubricant applicator device includes a tubular member to surround a portion of an endoscope insertion tube such that the tubular member is extendable over the portion of the endoscope insertion tube to snugly surround the portion of the endoscope insertion tube. An enlarged bulbous region adjacent to a distal end of the tubular member is pre-filled with a gel material that is releasable onto a surface area of the endoscope insertion tube encompassed by the enlarged bulbous region. A handle region adjacent to a proximal end of the tubular member that extends from the enlarged bulbous region provides a non-slip exterior surface to enable a human hand of an operator to grasp the handle region and manipulate the endoscope insertion tube.

### CLAIM OF PRIORITY

This patent application hereby incorporates by reference the entirety of the disclosures of the following cases and each of the cases on which they depend and further claims priority, incorporate by reference, and claims priority from :
a. U.S. Provisional Patent Application No. 62/430,932 titled “LUBRICANT APPLICATOR FOR ENDOSCOPE” filed on Dec. 7, 2016;
b. U.S. Provisional Patent Application No. 62/433,321 titled “VIEWING AID FOR ENDOSCOPE, AND METHOD” filed on Dec. 13, 2016; and
c. U.S. Provisional Patent Application No. 62/434,397 titled “AUTOMATING ENDOSCOPY INSTRUMENT INSERTION AND EXTRACTION THROUGH THE INSTRUMENT CHANNEL OF AN ENDOSCOPE” filed on Dec. 14, 2016.

### FIELD OF TECHNOLOGY

The disclosure relates broadly to medical devices and, more particularly to a system, a device, and a method of a lubricant applicator device to be used with an insertion tube of an endoscope.

### BACKGROUND

An endoscopy may be a procedure in which a body is examined internally using a medical device called an endoscope. An insertion tube of the endoscope may be a long, flexible tube which is inserted into a patient during the endoscopy. Before and during insertion, lubricant (e.g., a gel material) may be applied to the insertion tube. A doctor, nurse, and/or technician may have to manually apply the lubricant onto the insertion tube by placing the lubricant directly on the tube before each use of the endoscope. This may be a time consuming and repetitive process.

In addition, applying the lubricant may be a messy process (e.g., excess lubricant may drip onto a floor). The user of the endoscope may need to change gloves during each and every use of the insertion tube. The user's hands and clothing might get sticky and covered with lubricant. Another disadvantage of applying the lubricant multiple times during use of the endoscope may be an increased risk of contamination and a prolonged set up time. Further, during insertion of the endoscope and during subsequent procedures, it may also be important to grasp and manipulate a portion of the insertion tube which is external to the patient. This can be problematic if the portion of the insertion tube external to the patient is difficult to turn or is slippery.

### SUMMARY

Disclosed are a system, a device, and a method of a lubricant applicator device to be used with an insertion tube of an endoscope. In one aspect, a lubricant applicator system includes a tubular member, an enlarged bulbous region, and a handle region. The tubular member surrounds a portion of the endoscope insertion tube. The enlarged bulbous region is adjacent to a distal end of the tubular member. The handle region is adjacent to a proximal end of the tubular member. The tubular member is such that the tubular member is extendable (e.g., flexibly extendable, manipulatable, etc.) over the portion of the endoscope insertion tube to snugly surround the portion of the endoscope insertion tube. The enlarged bulbous region is pre-filled with a gel material that is releasable onto a surface area of the endoscope insertion tube encompassed by the enlarged bulbous region. The handle region adjacent to the proximal end of the tubular member extends from the enlarged bulbous region provides a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

A flow rate of the gel material onto the surface area of the endoscope insertion tube encompassed by the enlarged bulbous region may be proportional to the pressure applied to the enlarged bulbous region by the thumb of the human hand of the operator. The lubricant applicator device may be any one of a single use lubricant applicator device and/or a multiple use lubricant applicator device.

The gel material may be refillable in the enlarged bulbous region when the lubricant applicator device is the multiple use lubricant applicator device. The gel material may be pre-filled in the enlarged bulbous region at a factory when the lubricant applicator device is the single use lubricant applicator device.

The tubular member may be a single mold assembly including both the enlarged bulbous region and the handle region. The tubular member may be formed of a material including a rubber, a polymeric flexible material, a nylon material, a biodegradable material, a silicon based material, and/or a semi-rigid material. The insertion tube may be inserted into the lubricant applicator device axially through the proximal end of the tubular member and continuing through the tubular member. The insertion tube may emerge from the distal end of the tubular member adjacent to the enlarged bulbous region.

A section of the insertion tube encapsulated by the handle region may remain segregated from the gel material of the enlarged bulbous region such that an exterior surface of the handle region will remain unaffected by an extraction of the gel material from the enlarged bulbous region when the pressure of the thumb of the operator is applied on the enlarged bulbous region. The pressure of the thumb of the operator may cause the gel material to release onto the surface area of the endoscope insertion tube encompassed by the enlarged bulbous region of the lubricant applicator device.

The endoscope insertion tube may be transgressible through the lubricant applicator device when it is pushed through the tubular member from the proximal end of the tubular member. The gel material may be a water-soluble gel that is gentle on mucosal surfaces of a patient. The enlarged bulbous region may be a chamber that includes the gel material.

In another aspect, a lubricant applicator device includes a tubular member, an enlarged bulbous region and a handle region. The enlarged bulbous region is at a distal end of the lubricant applicator device. The handle region is at a proximal end of the lubricant applicator device. The tubular member surrounding a portion of an endoscope insertion tube is such that the tubular member is extendable (e.g., flexibly movable, manipulatable, stretchable, placeable, etc.) over the portion of the endoscope insertion tube to surround the portion of the endoscope insertion tube. The handle region provides a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

In yet another aspect, a method of forming a lubricant applicator device includes generating a single mold having a tubular member, an enlarged bulbous region, and a handle region. The enlarged bulbous region is at a distal end of the lubricant applicator device. The handle region is at a proximal end of the lubricant applicator device. The tubular member surrounding a portion of an endoscope insertion tube is such that the tubular member is placed over the portion of the endoscope insertion tube surrounding the portion of the endoscope insertion tube. The handle region provides a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

The lubricant applicator device may have a sideways water-tower like shape. The lubricant applicator device may be formed of a material including a rubber, a polymeric flexible material, a nylon material, a biodegradable material, a silicon based material, and/or a semi-rigid material.

The methods, devices, and systems disclosed herein may be implemented in any means for achieving various aspects, and may be executed in a form of a machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 is an apparatus view of a lubricant applicator device, according to an embodiment.

FIG. 2 is an endoscope view illustrating the environment of using the lubricant applicator device of FIG. 1, according to an embodiment.

FIG. 3 is a system view is a system view illustrating the environment to use the lubricant applicator device of FIG. 1, according to an embodiment.

FIG. 4 is an operation view explaining the working of the lubricant applicator device of FIG. 1, according to an embodiment.

FIG. 5 is a cross-sectional view of the lubricant applicator device of FIG. 1, according to an embodiment.

FIG. 6 is a gland view illustrating the gel chamber/gland at the inside of the lubricant applicator device of FIG. 1 and the chamber retaining wall for containing the gel at the ‘slice’, according to an embodiment.

Other features of the present embodiments will be apparent from the accompanying drawings and from the detailed description that follows.

### DETAILED DESCRIPTION

Disclosed are a system, a device, and a method of a lubricant applicator device to be used with an insertion tube of an endoscope. In the following description, for the purposes of explanation, numerous specific details are set forth in order to provide a thorough understanding of the various embodiments. It will be evident, however to one skilled in the art that the various embodiments may be practiced without these specific details.

An endoscope may be an instrument that can be introduced into the body to give a view of its internal parts. An endoscopy is a procedure where the inside of a body is examined using the endoscope. The endoscope may include an insertion tube that may have a light source and/or a camera at one end. Images of the inside of the body are relayed to a display screen. The insertion tube may be guided through (into) a body cavity via mechanisms controlled externally by a user. The insertion tube can be inserted into the body through a natural opening, such as into the mouth and down the throat, or through the bottom. The insertion tube can also be inserted through a small cut or incision made in the skin when keyhole surgery is being carried out.

The endoscopy can be used to investigate unusual symptoms, including but not limited to, difficulty swallowing (dysphagia), persistent abdominal pain, chest pain that is not caused by heart-related conditions, persistent nausea and vomiting, unexplained weight loss, vomiting blood, persistent diarrhea, and blood in stool. The endoscope can also help perform certain types of surgery, and/or remove a small sample of tissue for further analysis (e.g., a biopsy).

In one embodiment, a lubricant applicator device 150 includes a tubular member 100, an enlarged bulbous region 102, a handle region 104, a distal end 106, a proximal end 108, an insertion tube 110, a gel material 112, and an exterior non-slip surface 114. The tubular member 100 surrounds a portion of an endoscope insertion tube 110. The enlarged bulbous region 100 is adjacent to the distal end 106 of the tubular member 100, and the handle region 104 is adjacent to a proximal end 108 of the tubular member 100. The tubular member 100 is such that the tubular member 100 is extendable (e.g., flexibly extendable, manipulatable, etc.) over the portion of the endoscope insertion tube 110 to snugly surround the portion of the endoscope insertion tube 110. The enlarged bulbous region 102 is pre-filled with a gel material 112 that is releasable onto a surface area of the endoscope insertion tube 110 encompassed by the enlarged bulbous region 102. The handle region 104 is adjacent to the proximal end 108 of the tubular member 100. The tubular member 100 extends from the enlarged bulbous region 102 and provides a non-slip exterior surface 114 to enable a human hand 400 of an operator 320 to grasp the handle region 104 and manipulate the endoscope insertion tube 110 while rotatably affixing a thumb 402 of the hand onto the enlarged bulbous region 102 to release the gel material 112 when a pressure is applied to the enlarged bulbous region 102 using the thumb 402.

According to embodiments, a flow rate of the gel material 112 onto the surface area of the endoscope insertion tube 110 encompassed by the enlarged bulbous region 102 may be proportional to the pressure applied to the enlarged bulbous region 102 by the thumb 402 of the flexible portion 206 of the operator 320. The lubricant applicator device 150 may be any one of a single use lubricant applicator device 150 and/or a multiple use lubricant applicator device 150.

According to an embodiment, the gel material 112 may be refillable in the enlarged bulbous region 102 when the lubricant applicator device 150 is the multiple use lubricant applicator device 150. The gel material 112 may be pre-filled in the enlarged bulbous region 102 at a factory when the lubricant applicator device 150 is the single use lubricant applicator device 150.

According to embodiments, the tubular member 100 may be a single mold assembly including both the enlarged bulbous region 102 and the handle region 104. The tubular member 100 may be formed of a material including a rubber, a polymeric flexible material, a nylon material, a biodegradable material, a silicon based material, and/or a semi-rigid material. The insertion tube 110 may be inserted into the lubricant applicator device 150 axially through the proximal end 108 of the tubular member 100 and continuing through the tubular member 100 to emerge from the distal end 106 of the tubular member 100 adjacent to the enlarged bulbous region 100. Although particular materials for forming the tubular member 100 have been described, embodiments contemplate any number of materials to form the tubular member 100, according to particular needs.

A section of the insertion tube 110 encapsulated by the handle region 104 may remain segregated from the gel material 112 of the enlarged bulbous region 102 such that an exterior surface of the handle region 104 to remain unaffected by an extraction of the gel material 112 from the enlarged bulbous region 102 when the pressure of the thumb 402 of the operator 320 is applied on the enlarged bulbous region 102 causing the gel material 112 to release onto the surface area of the endoscope insertion tube 110 encompassed by the enlarged bulbous region 102 of the lubricant applicator device 150.

The endoscope insertion tube 110 may be transgressible through the lubricant applicator device 150 when it is pushed through the tubular member 100 from the proximal end 108 of the tubular member 100. The gel material 112 may be a water-soluble gel that is gentle on mucosal surfaces of a patient. The enlarged bulbous region 102 may be a chamber that includes the gel material 112.

In another embodiment, a lubricant applicator device 150 includes a tubular member 100 having an enlarged bulbous region 102 at a distal end 106 of the lubricant applicator device 150 and a handle region 104 at a proximal end 108 of the lubricant applicator device 150. The tubular member 100 surrounding a portion of an endoscope insertion tube 110 is such that the tubular member 100 is extendable (e.g., flexibly movable, manipulatable, stretchable, placeable, etc.) over the portion of the endoscope insertion tube 110 to surround the portion of the endoscope insertion tube 110. The handle region 104 provides a non-slip exterior surface 114 to enable a flexible portion 206 of an operator 320 to grasp the handle region 104 and manipulate the endoscope insertion tube 110 while rotatably affixing a thumb 402 of the hand onto the enlarged bulbous region 102 to release the gel material 112 when a pressure is applied to the enlarged bulbous region 102 using the thumb 402.

A flow rate of the gel material 112 onto the surface area of the endoscope insertion tube 110 encompassed by the enlarged bulbous region 102 may be proportional to the pressure applied to the enlarged bulbous region 102 by the thumb 402 of the flexible portion 206 of the operator 320. The lubricant applicator device 150 may be any one of a single use lubricant applicator device 150 and/or a multiple use lubricant applicator device 150.

The gel material 112 may be refillable in the enlarged bulbous region 102 when the lubricant applicator device 150 is the multiple use lubricant applicator device 150. The gel material 112 may be pre-filled in the enlarged bulbous region 102 at a factory when the lubricant applicator device 150 is the single use lubricant applicator device 150.

In yet another embodiment, a method of forming a lubricant applicator device 150 includes generating a single mold having a tubular member 100 with an enlarged bulbous region 102 at a distal end 106 of the lubricant applicator device 150 and a handle region 104 at a proximal end 108 of the lubricant applicator device 150. The tubular member 100 surrounding a portion of an endoscope insertion tube 110 is such that the tubular member 100 is placed over the portion of the endoscope insertion tube 110 surrounding the portion of the endoscope insertion tube 110. The handle region 104 provides a non-slip exterior surface 114 to enable a flexible portion 206 of an operator 320 to grasp the handle region 104 and manipulate the endoscope insertion tube 110 while rotatably affixing a thumb 402 of the hand onto the enlarged bulbous region 102 to release the gel material 112 when a pressure is applied to the enlarged bulbous region 102 using the thumb 402.

The lubricant applicator device 150 may have a sideways water-tower like shape. The lubricant applicator device 150 may be formed of a material including a rubber, a polymeric flexible material, a nylon material, a biodegradable material, a silicon based material, and/or a semi-rigid material. The lubricant applicator device 150 may be any one of a single use lubricant applicator device 150 and/or a multiple use lubricant applicator device 150. The gel material 112 may be refillable in the enlarged bulbous region 102 when the lubricant applicator device 150 is the multiple use lubricant applicator device 150. The gel material 112 may be pre-filled in the enlarged bulbous region 100 at a factory when the lubricant applicator device 150 is the single use lubricant. Although particular materials for forming the lubricant applicator device 150 have been described, embodiments contemplate any number of materials to form the lubricant applicator device 150, according to particular needs.

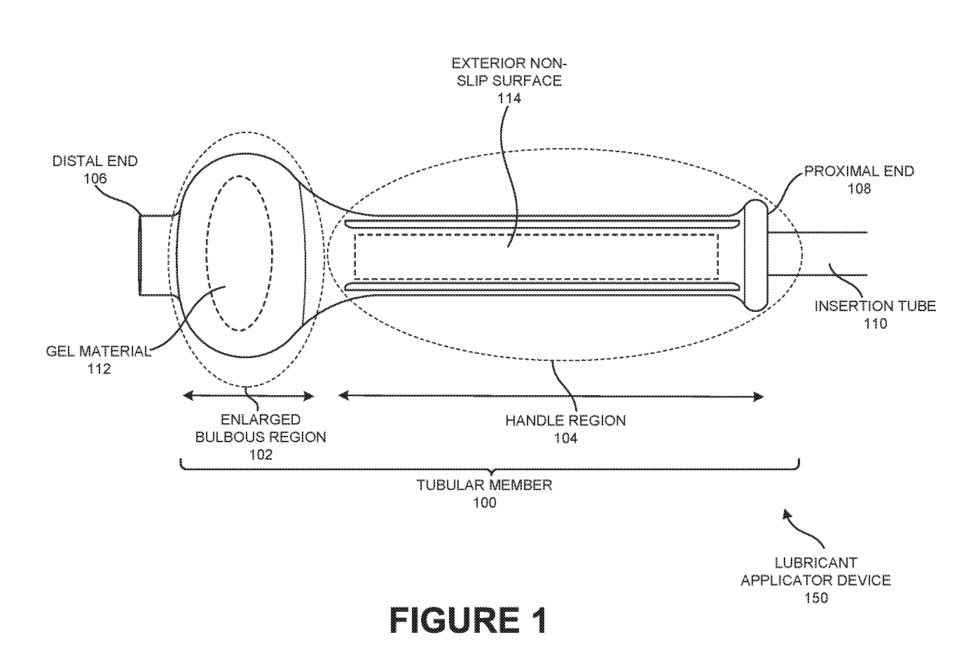

FIG. 1 is an apparatus view of a lubricant applicator device, according to an embodiment. Lubricant applicator device 150 illustrating different parts of the lubricant applicator device 150, according to one or more embodiments. Particularly, FIG. 1 illustrates a tubular member 100, an enlarged bulbous region 102, a handle region 104, a distal end 106, a proximal end 108, an insertion tube 110, a gel material 112, and an exterior non-slip surface 114 of the lubricant applicator device 150, according to one or more embodiments.

Lubricant applicator device 150 may comprise an elongated, generally tubular (or cylindrical) element (or member) 100, having two ends (e . g ., proximal end 108, distal end 106). One end (e . g ., proximal end 108) may be referred to as the “doctor end” or “back end” or “proximal end” of the tubular element 100. The other end (e.g., distal end 106) may be referred to as the “patient end” or “front end” or “distal end” of the tubular element 100, according to one embodiment.

The tubular element 100 may be made of a flexible rubber (or other elastomer) material, by a molding process. Some suitable materials for the tubular element 100 may include silicon rubber, or other polymeric flexible or semi-rigid material, such as selected from the group consisting of polydimethylsiloxane (PDMS), PMMA (poly(methyl methacrylate)), PS (polystyrene), and PC (polycarbonate). Other materials may include silicon, vitron rubber, and nylon, according to one embodiment. Although particular materials forming the tubular element 100 have been described, embodiments contemplate any number of materials to form the tubular element 100, according to particular needs.

The tubular element 100 be may have a length of approximately four (4) inches (200 mm), and an inner diameter of approximately ⅝ inches (15 mm). The inner diameter of the tubular element 100 may be slightly smaller than the outer diameter of the insertion tube 110, so that it may securely grip or hug the insertion tube 110, according to an embodiment.

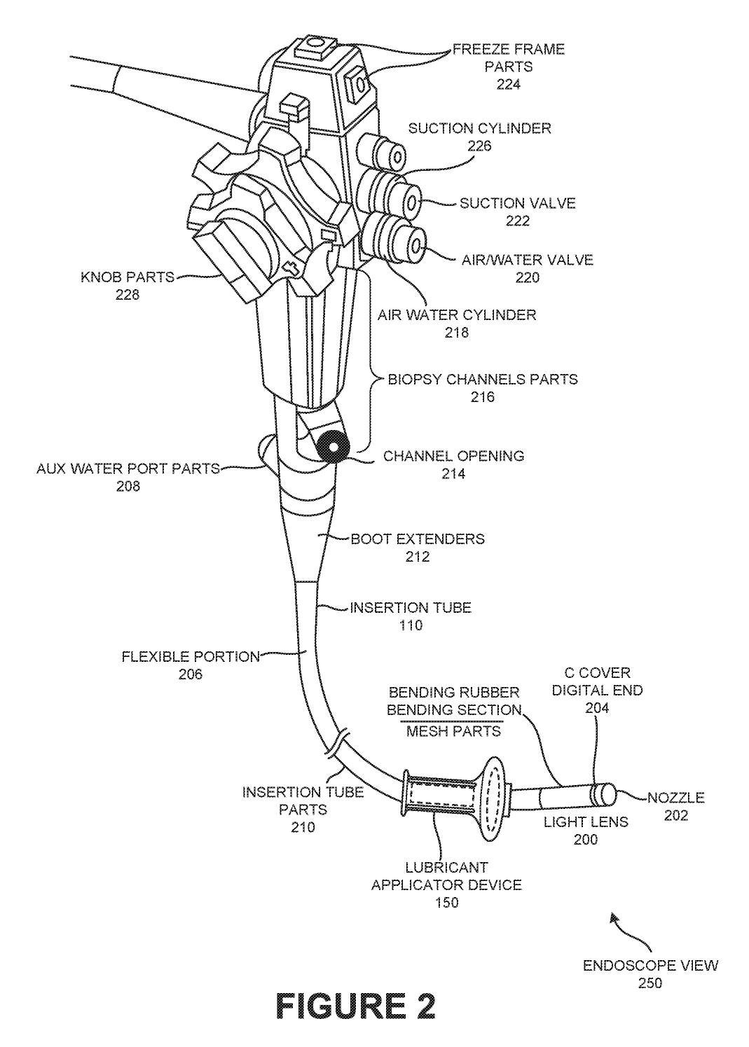

FIG. 2 is an endoscope 250 illustrating a view of the environment of using the lubricant applicator device 150 of FIG. 1 while performing an endoscopy, according to an embodiment. It is noted that the lubricant applicator device 150 must be designed and calibrated to ensure that it does not interfere with a proper operation of the complex operation of the endoscope 250 as described herein. Particularly, FIG. 2 builds on FIG. 1, and further adds a light lens 200 a nozzle 202, a c cover digital end 204, a flexible portion 206, an aux water port 208, an insertion tube 210, one or more boot extenders 212, a channel opening 214, one or more biopsy channels 216, an air water cylinder 218, air/water valve 220, a suction valve 222, one or more freeze frame parts 224, a suction cylinder 226, and one or more knob parts 228, according to one or more embodiments. The light lens 200 may enable illumination of a target object inside a body. Other items described as 202-228 may be components of the lubricant applicator. Illustrated specifically in FIG. 2 the placement of the lubricant applicator device 150 on a portion of the insertion tube 210.

The knob parts 228 may be part of the control body of the endoscope 250. This section of the scope may contain the physician controls: angulation control knobs, forceps raiser or elevator lifter, air/water, suction control valves, remote video switches, biopsy port and focus mechanism for fiber optic scopes, tension controller and the control body for holding the endoscope.

The insertion tube 110 of FIG. 1, as illustrated in FIG. 2, may be a long flexible tube inserted into the body during normal use. The length and diameter of the insertion tube 110 may greatly by model as is dependent upon the anatomy viewed. Similar to the light guide tube, it may be constructed of multiple layers of steel coil, fiber mesh and vulcanized rubber. The thickness of each layer may vary by model and by tube diameter. A clear protective outer layer may be labeled with metric insertion depth marks.

The insertion tube 110 may be hollow and may provide protection for the delicate internal components including the angulation system, fiber optics, video signal wires and biopsy channel. Sharp bending and over-coiling of the insertion tube may cause damage to the tube itself, and may damage the internal components. The damage may often indicated by raised ridges or buckles appearing along the tube.

Bending Section—Located at the distal end of the insertion tube 110, the bending section may be constructed of articulating steel ribs riveted together to form a flexible skeleton-like shell. The shell may be covered with a steel mesh braid and then a thin-walled flexible tube called the bending rubber or sheath. The distal end of the bending section may contain the video chip, optical glass lens systems, fiber optics and/or the termination nozzle of the air/water system. The overall length of the bending section may vary by model but it is usually around four inches long. The outside diameter of most endoscope 250 variants such as gastroscopes, duodenoscopes, and colonoscopes can range from 5.9 to 12.5 mm.

The bending section is the most fragile portion of the endoscope 250 and must be protected from impact. Impact to the distal tip potentially may damage all of the inner components.

The endoscope 250 may have a light transmission component for image illumination. The light may enter the scope, usually at the light guide connector, through the light guide prong and/or paddle connector. The endoscope 250 may have a self-contained battery-powered light source for use when access to a cart system is impractical.

Inside the endoscope 250, light may travel from the light guide prong to the distal end through a light fiber bundle, a collection of thousands of thin, flexible optical glass fibers. Each individual fiber may transport a small portion of light, and together they can produce a very intense bright light. The light may exit the endoscope 250 at the distal end through the light guide lenses, which disperse the light evenly across the visual field. Typical GI scope variants of the endoscope 250 may have two or three light guide bundles.

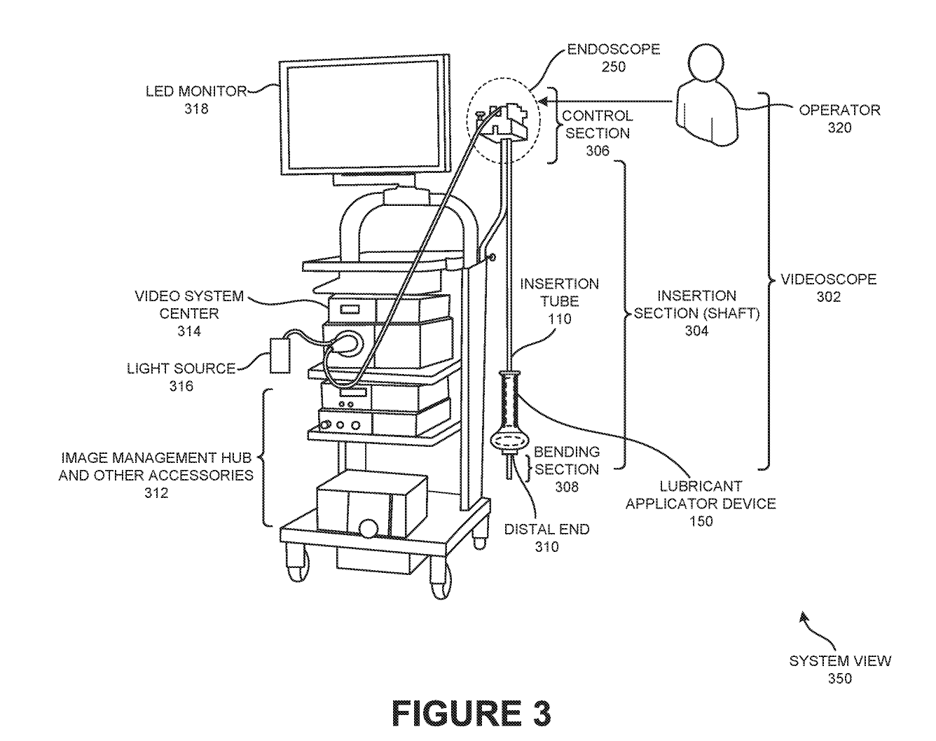

There is no adjustment capacity within the light fiber system in the scope. All light adjustments are made manually on the light source or automatically within the video cart system (e.g., the system view 350 of FIG. 3 may be a video cart system). Damage to the fibers results in reduced light transmission.

The primary function of the endoscope 250 may be imaging. An image may be captured at the distal end (using the objective lens system) and is then transferred to the image transmission device directly behind the lens system. A variant of the endoscope 250 may be a fiber optic endoscope. Fiber optic endoscope variants may use an image fiber bundle (similar to the light fiber bundle) to carry the image to an ocular lens at the control body for viewing. Each of the thousands of fibers may carry a small portion of the image, and a black dot on the image represents one broken fiber.

Video endoscopes (videoscopes) may also be a variant of the endoscope 250. On video endoscope embodiment, the video chip, a charge coupled device (or CCD) is located directly behind the objective lens assembly at the distal end. Such systems may use a CMOS video chip (e.g., complementary metal oxide semiconductor) Here the image may be converted to a digital signal. It may travel through signal wires running through the scope to the light guide connector or video connector. The digital signal then may connect directly to and/or through a cable to the video processor on the cart system. The image can be sent to a variety of monitors, printers or remote locations. The Thinking Cap may provide a visual image of a yellow submarine to illustrate this process. Endoscope video chips may contain from 100,000 to 1 million pixels. More pixels usually may mean better image quality, resolution and color reproduction.

The endoscope 250 may have an angulation system of wires, guided by knobs on the control body. This system may manipulate the bending section for insertion and viewing. The knobs may be connected inside the control body housing to either a pulley-and-wire system or a gear-and-chain system that connects to the angle wires. The angle wires may be braided stainless steel cables that extend from the control body through the insertion tube and connect to the bending ribs at the distal end. Each wire may control one direction of movement, so two opposite wires work together to provide movement up and down, left and right. The angle wires inside the insertion tube may be encased within a protective coil pipe that protects the other internal components from the angle wire.

Some colonoscope models of the endoscope 250 may contain an additional wiring component to boost the rigidity of the insertion tube. This rigor may be provided in three levels and is designed to be neutralized before withdrawing the colonoscope after terminal intubation has been achieved. Excess wear and tear on this wired enhancement may be minimized when the stiffness is deactivated for transport, decontamination, reprocessing and storage.

The endoscope 250 may include a biopsy/suction system. The biopsy and suction channels may be part of the same internal system. The biopsy channel may be a straight piece of uniform flexible tubing running from the distal end to the control body. The suction channel may be similar but may run from the control body to the light guide connector. The suction and biopsy channels may be joined in the control body and are controlled by the suction valve controls.

An external suction device connected to the suction channel at the light guide connector may provide the suction pressure needed to perform biopsy, and suctioned material then may travel back through the biopsy channel at the biopsy port opening. The endoscope 250 may include an Air/Water System. The air and water internal channels may be thin flexible tubes running from the light guide connector to the distal end. Although air and water flow through separate tubes, they may be controlled by the same valve on the control body. Air and water exit the scope at the distal end through the air/water nozzles, which may direct the flow over the objective lens.

The endoscope 250 may include a forcep raiser function. The forcep raiser (e.g., elevator) may be at the distal end to deflect accessories passed through the biopsy channel. This small metal arm at the biopsy port opening may be attached to a wire that runs inside the insertion tube, connects to a control lever on the control body, and operates much like the angulation system controls. The forceps raising function may be found on duodenoscopes and endoscopic ultrasound scope variants of the endoscope 250.

Except for the electrical video connections, the entire outer surface of the endoscope 250 may be chemical and fluid resistant. Any seams between parts may be sealed by O-rings, and a colonoscope may contain over forty of these O-rings. The chemical- and fluid-resistant coatings on the flexible tubes may themselves flexible, and some parts of the endoscope 250 may be sealed with epoxy. The ends of the bending rubber may be sealed with silk or nylon braid and then covered with epoxy. Caps or covers may be provided to seal the electrical video connections against fluid. The end result is a endoscope device 250 that, when undamaged, can be immersed completely in water or cleaning/disinfecting fluid. In this way, the instrument can be reprocessed over and over.

FIG. 3 is a system view 350 illustrating the environment to use the lubricant applicator device 150 of FIG. 1, according to an embodiment. The lubricant applicator device 150 is connected to the endoscope 250 for the operator 320 to be able to see the inside of human body on an LED monitor 318 while endoscopy, according to an embodiment. Particularly, FIG. 3 builds on FIGS. 1 and 2, and further adds, a videoscope 302, an insertion section (shaft) 304, a control section 306, a bending section 308, the distal end 106, an image management hub and other accessories 312, a video system center 314, a light source 316, an LED monitor 318, and an operator 320, according to one embodiment. The various components 302-318 may be used in a data processing system that controls the endoscope 250 illustrated in FIG. 3. The endoscope 250 in FIG. 3 is illustrated as utilizing the lubricant applicator device 150 of FIG. 1.

While performing an endoscopy, the operator 320 may be able to operate the endoscope 250 to investigate the inside of the human body using control section 306. The lubricant applicator device 150 may be connected to the endoscope 250 through the insertion tube 110. The endoscope 250 may be connected to an image management hub 312 for a real-time display of endoscopy on the LED monitor 318.

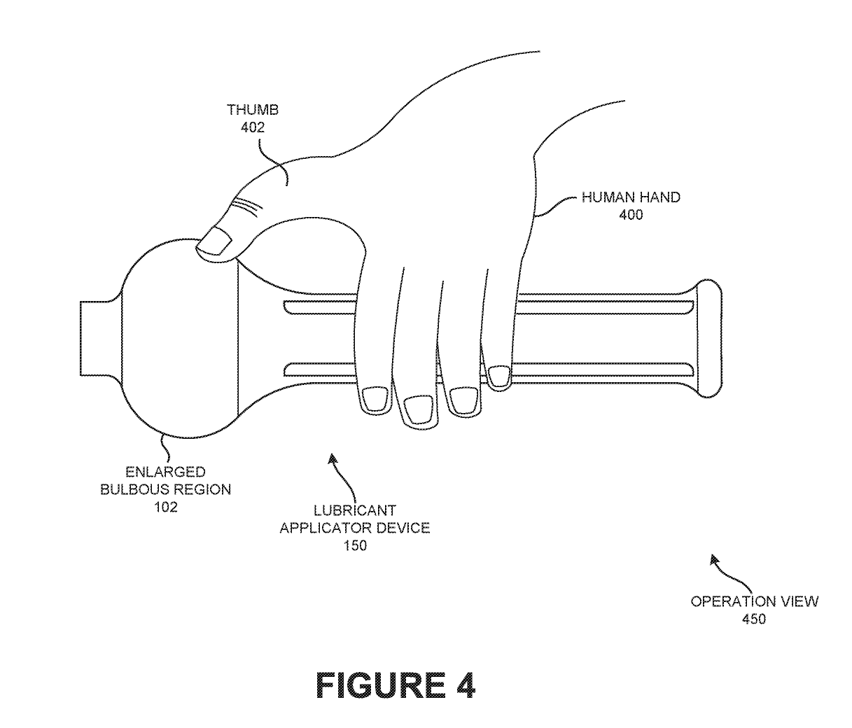

FIG. 4 is an operation view 450 explaining the working of the lubricant applicator device 150 of FIG. 1, according to an embodiment. The enlarged bulbous region 102 of the tubular element 100 may contain gel material 112. The operator 320 may squeeze the enlarged bulbous region 102 of the tubular element 100 by pressing the thumb 402 onto the insertion tube 110 to dispense the gel material 112, according to one embodiment.

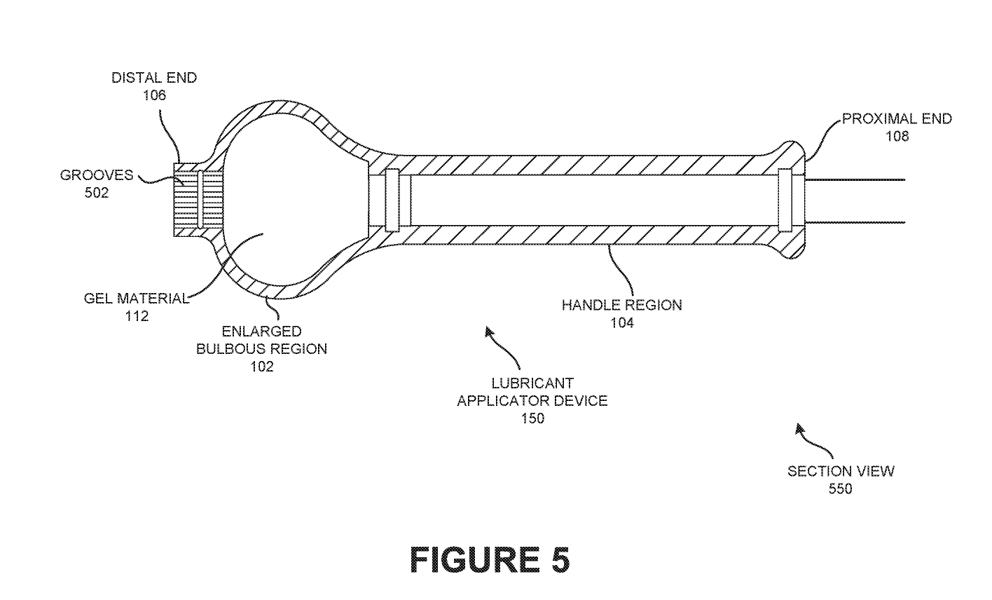

FIG. 5 is a cross-sectional view of the lubricant applicator device 150 of FIG. 1 showing the inside of the lubricant applicator device 150, according to an embodiment.

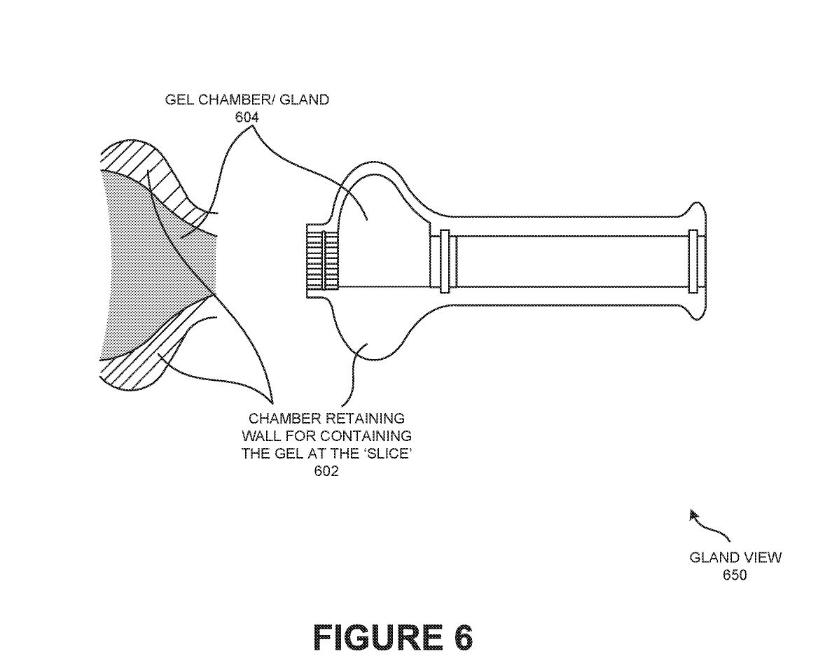

FIG. 6 is a gland view 650 illustrating the gel chamber/gland 604 at the inside of the lubricant applicator device of FIG. 1 and the chamber retaining wall for containing the gel at the ‘slice’ 602, according to an embodiment.

According to an embodiment of FIG. 6, the enlarged bulbous region 102 of the lubricant applicator device 150 at the distal end 106 of the tubular element 100, may be enlarged and have a larger diameter than the remainder of the tubular element 100, to contain the gel material 112 at the chamber retaining wall at the “slice” 602. This portion (as shown in enlarged bulbous region 102) of the tubular element 100 may be referred to as a “gel chamber”, or “gland”, or “chamber”, or “bulb” (as shown in gel chamber/gland 604). When in use, the gel material 112 may be dispensed and/or excreted onto the insertion tube 110 by squeezing or pressing the gel chamber/gland 604, according to one embodiment.

According to one embodiment, FIG. 2 is a diagram of an endoscope (as shown in endoscope view 250). The endoscope (e.g., endoscope view 250) may comprise a long flexible insertion tube 110 having two ends. The insertion tube may have an outside diameter (OD) of approximately ⅝″ (15 mm). An instrument channel may extend through the extension tube from control apparatus (e.g., control section 306) disposed at a proximal (user, doctor) end 108 of the insertion tube to the distal (patient) end 106 of the insertion tube, according to one embodiment.

An end cap or nozzle 202 may be disposed at the distal end 106 of the insertion tube 110, and may contain lenses, water jets, etc. (not shown). A distal end 106 portion of the insertion tube 110 may be flexible, and have an irregular surface. In use, it may be beneficial that the insertion tube 110 be lubricated (with a gel material 112), without contaminating or clogging the lenses, water jets, etc., according to one embodiment.

FIG. 1 is a diagram illustrating an embodiment of a lubricant applicator (e.g., as shown in lubricant applicator device 150). The applicator may be referred to as the EndoGrip™ applicator, according to one embodiment.

The applicator (e.g., lubricant applicator device 150) may comprise an elongated, generally tubular (or cylindrical) element (or member) 100, having two ends (e . g ., proximal end 108, distal end 106). One end (e . g ., proximal end 108) may be referred to as the “doctor end” or “back end” or “proximal end” of the tubular element 100. The other end (e.g., distal end 106) may be referred to as the “patient end” or “front end” or “distal end” of the tubular element 100, according to one embodiment.

The tubular element 100 may be made of a flexible rubber (or other elastomer) material, by a molding process. Some suitable materials for the tubular element 100 may include silicon rubber, or other polymeric flexible or semi-rigid material, such as selected from the group consisting of polydimethylsiloxane (PDMS), PMMA (poly(methyl methacrylate)), PS (polystyrene), and PC (polycarbonate). Other materials may include silicon, vitron rubber, and nylon, according to one embodiment.

The tubular element 100 be may have a length (L) of approximately 4 inches (200 mm), and an inner diameter (ID) of approximately ⅝ inches (15 mm). The inner diameter (ID) of the tubular element 100 may be slightly smaller than the outer diameter of the insertion tube 110, so that it may securely grip (or “hug”) the insertion tube 110, according to one embodiment.

According to one embodiment of FIG. 1, the tubular element 100 may be disposed over the insertion tube 110.

In use, the insertion tube 110 may be inserted, axially, through the tubular element 100, from the back end (e.g., as shown in proximal end 108) thereof, continuing through the tubular element 100, and emerging from the front end (e.g., as shown in distal end 106) of the tubular element 100. Alternatively (vice-versa), the tubular element 100 may be disposed, axially, over the insertion tube 110 by pushing the back end (e.g., as shown in proximal end 108) of the tubular element 100 over the front end (e.g., distal end 106) of the insertion tube 110.

According to an embodiment of FIG. 6, a bulbous portion (e.g., as shown in enlarged bulbous region 102), such as a portion at the front end (e.g., as shown in distal end 106) of the tubular element 100, may be enlarged (have a larger diameter than the remainder of the tubular element 100), to contain a quantity, such as 75 cc, of lubricant (e.g., gel material 112). This portion (e.g., as shown in enlarged bulbous region 102) of the tubular element 100 may be referred to as a “gel chamber”, or “gland”, or “chamber”, or “bulb” (e.g. as shown in gel chamber/gland 604). In use, gel (e.g., gel material 112) may be dispensed (or excreted) onto the insertion tube 110 by squeezing (or pressing) the bulb (e.g. as shown in gel chamber/gland 604), according to one embodiment.

The outside surface of the remaining cylindrical (or grip) portion (e.g., as shown in handle region 104) of the tubular element 100 may be provided with ridges, dappled, or the like to improve gripping (e.g., a bicycle hand grip), when the tubular element 100 is installed on the insertion tube 110. The inside surface of the grip portion (e.g., as shown in handle region 104) may also be provided with ridges, dappled, or the like to improve gripping of the insertion tube 110. This is advantageous because the insertion tube 110 has a relatively small diameter (⅝″), making it somewhat difficult to manipulate (particularly if it is lubricated). It may be easier for the user (e.g., operator 320) to manipulate the insertion tube 110 via the tubular element (or grip) 100 which may have a grip (e.g., non-bulbous portion, handle region 104) diameter of approximately 1 ¼ inches (30 mm), according to one embodiment.

While the invention(s) has/have been described with respect to a limited number of embodiments, these should not be construed as limitations on the scope of the invention(s), but rather as examples of some of the embodiments. Those skilled in the art may envision other possible variations, modifications, and implementations that are also within the scope of the invention(s), and claims, based on the disclosure(s) set forth herein, according to one embodiment.

What is claimed is:

1. A lubricant applicator device, comprising:
a tubular member to surround a portion of an endoscope insertion tube such that the tubular member is extendable over the portion of the endoscope insertion tube to snugly surround the portion of the endoscope insertion tube;
an enlarged bulbous region adjacent to a distal end of the tubular member to be pre-filled with a gel material that is releasable onto a surface area of the endoscope insertion tube encompassed by the enlarged bulbous region; and
a handle region adjacent to a proximal end of the tubular member that extends from the enlarged bulbous region to provide a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

2. The lubricant applicator device of claim 1 wherein a flow rate of the gel material onto the surface area of the endoscope insertion tube encompassed by the enlarged bulbous region is proportional to the pressure applied to the enlarged bulbous region by the thumb of the human hand of the operator.

3. The lubricant applicator device of claim 1 wherein the lubricant applicator device is any one of a single use lubricant applicator device and a multiple use lubricant applicator device.

4. The lubricant applicator device of claim 3 wherein the gel material is refillable in the enlarged bulbous region when the lubricant applicator device is the multiple use lubricant applicator device.

5. The lubricant applicator device of claim 3 wherein the gel material is pre-filled in the enlarged bulbous region at a factory when the lubricant applicator device is the single use lubricant applicator device.

6. The lubricant applicator device of claim 1 wherein the tubular member is a single mold assembly comprising of both the enlarged bulbous region and the handle region and is formed of a material comprising at least one of a rubber, a polymeric flexible material, a nylon material, a biodegradable material, a silicon based material, and a semi-rigid material.

7. The lubricant applicator device of claim 1 wherein the insertion tube is inserted into the lubricant applicator device axially, through the proximal end of the tubular member and continuing through the tubular member to emerge from the distal end of the tubular member adjacent to the enlarged bulbous region.

8. The lubricant applicator device of claim 1 wherein a section of the insertion tube encapsulated by the handle region to remain segregated from the gel material of the enlarged bulbous region such that an exterior surface of the handle region to be unaffected by an extraction of the gel material from the enlarged bulbous region when the pressure of the thumb of the operator is applied on the enlarged bulbous region causing the gel material to release onto the surface area of the endoscope insertion tube encompassed by the enlarged bulbous region of the lubricant applicator device.

9. The lubricant applicator device of claim 1 wherein the endoscope insertion tube is transgressible through the lubricant applicator device when it is pushed through the tubular member from the proximal end of the tubular member.

10. The lubricant applicator device of claim 1 wherein the gel material is a water-soluble gel that is gentle on mucosal surfaces of a patient.

11. The lubricant applicator device of claim 1 wherein the enlarged bulbous region is a chamber that comprises the gel material.

12. A lubricant applicator device, comprising:
a tubular member having an enlarged bulbous region at a distal end of the lubricant applicator device and a handle region at a proximal end of the lubricant applicator device,
wherein the tubular member to surround a portion of an endoscope insertion tube such that the tubular member is flexible over the portion of the endoscope insertion tube to surround the portion of the endoscope insertion tube, and
wherein a handle region to provide a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

13. The lubricant applicator device of claim 12 wherein a flow rate of the gel material onto the surface area of the endoscope insertion tube encompassed by the enlarged bulbous region is proportional to the pressure applied to the enlarged bulbous region by the thumb of the human hand of the operator.

14. The lubricant applicator device of claim 12 wherein the lubricant applicator device is any one of a single use lubricant applicator device and a multiple use lubricant applicator device.

15. The lubricant applicator device of claim 14 wherein the gel material is refillable in the enlarged bulbous region when the lubricant applicator device is the multiple use lubricant applicator device.

16. The lubricant applicator device of claim 14 wherein the gel material is pre-filled in the enlarged bulbous region at a factory when the lubricant applicator device is the single use lubricant applicator device.

17. A method of forming a lubricant applicator device, comprising:
generating a single mold having a tubular member with an enlarged bulbous region at a distal end of the lubricant applicator device and a handle region at a proximal end of the lubricant applicator device,
wherein the tubular member to surround a portion of an endoscope insertion tube such that the tubular member is placeable over the portion of the endoscope insertion tube to surround the portion of the endoscope insertion tube, and
wherein the handle region to provide a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

18. The method of claim 17 wherein the lubricant applicator device has a sideways water-tower like shape.

19. The method of claim 17 wherein the lubricant applicator device is formed of a material comprising at least one of a rubber, a polymeric flexible material, a nylon material, a biodegradable material, a silicon based material, and a semi-rigid material.

20. The method of claim 17 wherein the lubricant applicator device is any one of a single use lubricant applicator device and a multiple use lubricant applicator device,
wherein the gel material is refillable in the enlarged bulbous region when the lubricant applicator device is the multiple use lubricant applicator device, and
wherein the gel material is pre-filled in the enlarged bulbous region at a factory when the lubricant applicator device is the single use lubricant applicator device.
